(19) United States
(12) Patent Application Publication
(10) Pub. No.: US 2024/0226305 A1
(43) Pub. Date: Jul. 11, 2024

(54) COMPOUNDS FOR TREATMENT OF TYPE 2 DIABETES

(71) Applicants: David R. ELMALEH, Newton, MA (US); Seetharamaiyer PADMANABHAN, Ashburn, VA (US); Timothy M. SHOUP, Franklin, MA (US)

(72) Inventors: David R. ELMALEH, Newton, MA (US); Seetharamaiyer PADMANABHAN, Ashburn, VA (US); Timothy M. SHOUP, Franklin, MA (US)

(21) Appl. No.: 18/540,091

(22) Filed: Dec. 14, 2023

Related U.S. Application Data
(60) Provisional application No. 63/387,533, filed on Dec. 15, 2022.

(51) Int. Cl. A61K 47/55; A61K 47/54; A61P 3/04; A61P 3/10

(52) U.S. Cl. CPC A61K 47/55; A61K 47/542; A61K 47/545; A61P 3/04; A61P 3/10

(57) ABSTRACT

This invention relates to dual activity compounds and uses thereof for the treatment of type 2 diabetes in patients who are overweight or obese.

### CROSS REFERENCE TO RELATED APPLICATIONS

This application claims the benefit of U.S. Provisional Patent Application Ser. No. 63/387,533, filed Dec. 15, 2022, which is incorporated herein by reference in its entirety.

### FIELD OF THE INVENTION

This invention relates to dual activity compounds and uses thereof for treatment of type 2 diabetes in patients who are overweight or obese.

### BACKGROUND OF THE INVENTION

Despite the large number of available drug options to treat type 2 diabetes (T2D), significant unmet need remains for more efficacious therapies that have added beneficial effects, particularly weight loss, as obesity rates are on the rise globally.

Metformin suppresses gluconeogenesis and improves glucose uptake and insulin sensitivity. It is considered the mainstay of T2D therapy for non-insulin treated patients, both as monotherapy and combination therapy. Approximately, all non-insulin treated patients (˜70% of all T2D patients) receive metformin.

Fenfluramine acts as a serotonin releasing agent, agonist of the serotonin 5-HT2 receptors, and σ1 receptor positive modulator. It is safe and used for managing exogenous obesity as a short-term adjunct in a regimen of weight reduction based on caloric restriction.

The present invention provides a first-in-class example of compounds with a dual activity mechanism poised to address the unmet need for overweight/obsess T2D patients (˜90% of T2D patients are overweight (˜30%) or obese (˜60%)) with balancing glucose uptake and insulin sensitivity and reducing appetite and inducing weight loss.

### SUMMARY OF THE INVENTION

In one aspect, the invention provides a compound represented by formula (I)

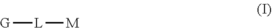

wherein
G is NRₐR_{b}—C(NH)—, a fenfluramine moiety, or a serotonin moiety;
M is —NH—C(NH)—NH—C(NH)—N(CH₃)₂;
L is a linker selected from the group consisting of a bond, —C(O)—(CH₂)ₘT-(CH₂)ₙ—C(O)—, -T-C(O)—CH₂—CH₂—C(O)—, —C(O)—CH₂—CH₂—C(O)-T-, —C(O)—CH₂—CH₂—C(O)-T-C(O)—CH₂—CH₂—C(O)—, and a 3-cyclobutene-1,2-dione moiety;
T is a bond, —X₁—CH₂—CH₂—X₂—, an amino acid moiety, a heterocycloalkyl moiety, a hydroxybenzoyl moiety, or an aminophenol moiety;
X₁ and X₂ are each O or NR_{c};
Rₐ, R_{b}, and R_{c} are each independently H, an alkyl group, or an acyl group, and
m and n are each 1, 2, or 3,
   or a pharmaceutically acceptable salt thereof.

In some embodiments, the compound of formula (I) is represented by a compound of formula (II)

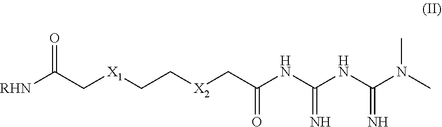

wherein RNH is a fenfluramine moiety or a serotonin moiety.

In some embodiments, the compound of formula (I) is represented by a compound of formula (III)

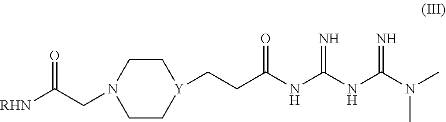

wherein RNH is a fenfluramine moiety or a serotonin moiety, and wherein Y is N or CH.

In some embodiments, the compound of formula (I) is represented by a compound of formula

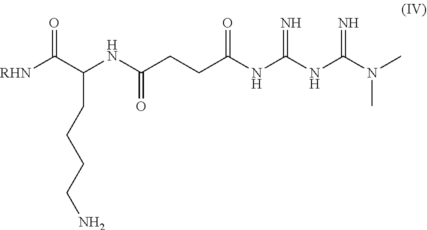

wherein RNH is a fenfluramine moiety or a serotonin moiety.

In some embodiments, the compound of formula (I) is represented by a compound of formula (V)

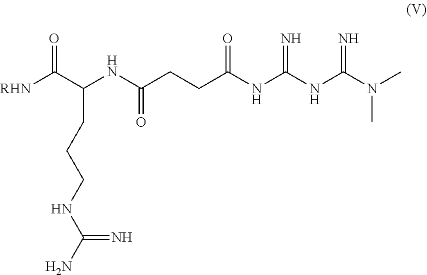

wherein RNH is a fenfluramine moiety or a serotonin moiety.

In one aspect, the present invention provides a pharmaceutical composition comprising a compound of the invention as described herein and a pharmaceutically acceptable carrier.

In another aspect, the present invention provides a method of treating diabetes in a subject in need thereof, comprising administering to the subject a compound of the invention as described herein.

In another aspect, the present invention provides a method of treating obesity or inducing weight reduction in a subject in need thereof, comprising administering to the subject a compound of the invention as described herein.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 depicts an HPLC spectrum of N,N-dimethyl triguanidine.

FIG. 2 depicts an HPLC spectrum of N,N-dimethyl triguanidine co-injected with metformin.

FIG. 3 provides Table 2 showing animal glucose measurement data (mg/dL).

FIG. 4 depicts blood glucose level results.

FIG. 5 depicts glucose tolerance test (OGTT 2 g/kg).

FIG. 6 provides Table 3 showing measurement data.

FIG. 7 depicts B. weight results.

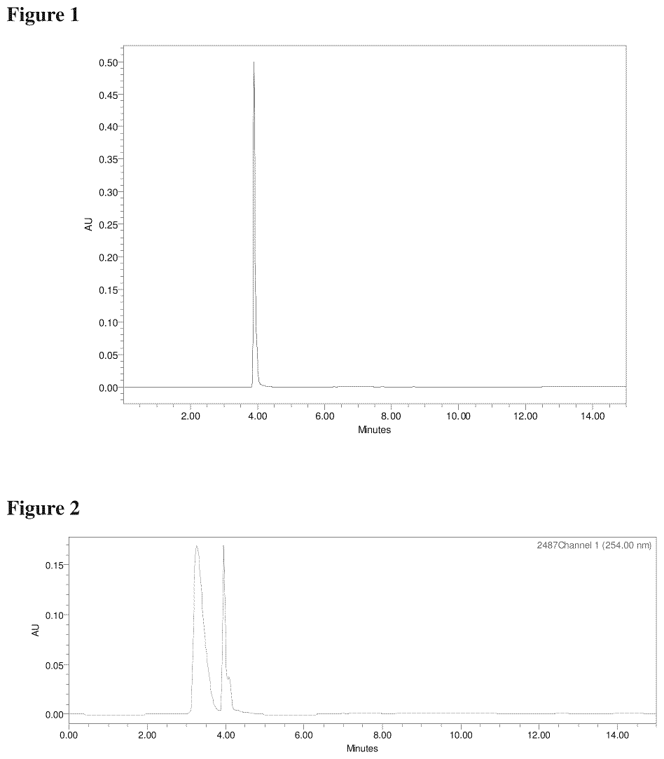

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention relates to a dual acting molecular structure combination of two treatment approaches that has demonstrated significant HbAlc and weight reduction in T2D animal model.

The novel molecular structure has shown several advantages over a simple combination of metformin and fenfluramine in preclinical studies. It is expected to be safe, have better glycemic control, induce weight loss (up to 5%) in short-term, improve overall mood, and enhance health-related quality of life. Fenfluramine (serotonin/dopamine), metformin (guanidine-based) and linkers for T2D and obesity drugs.

In one aspect, the invention provides a compound represented by formula (I)

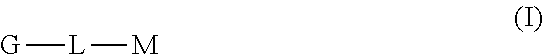

wherein
G is NRₐR_{b}—C(NH)—, a fenfluramine moiety, or a serotonin moiety;
M is —NH—C(NH)—NH—C(NH)—N(CH₃)₂;
L is a linker selected from the group consisting of a bond, —C(O)—(CH₂)ₘ-T-(CH₂)ₙ—C(O)—, -T-C(O)—CH₂—CH₂—C(O)—, —C(O)—CH₂—CH₂—C(O)-T- , —C(O)—CH₂—CH₂—C(O)-T-C(O)—CH₂—CH₂—C(O)—, and a 3-cyclobutene-1,2-dione moiety;
T is a bond, —X₁—CH₂—CH₂—X₂—, an amino acid moiety, a heterocycloalkyl moiety, a hydroxybenzoyl moiety, or an aminophenol moiety;
X₁ and X₂ are each O or NR_{c};
Rₐ, R_{b}, and Re are each independently H, an alkyl group, or an acyl group, and
m and n are each 1, 2, or 3,
   or a pharmaceutically acceptable salt thereof.

In some embodiments of the compound of the invention, L is a bond.

In some embodiments, L is —C(O)—CH₂-CH₂—C(O)—.

In some embodiments, L is a 3-cyclobutene-1,2-dione moiety.

In some embodiments, L is —C(O)—(CH₂)ₘ-T-(CH₂)ₙ—C(O)—.

In some embodiments, m is 1 and n is 1.

In some embodiments of the compound of the invention, L is

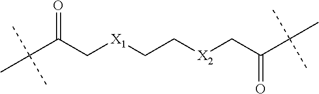

In some embodiments, X₁ and X₂ are O.

In some embodiments, X₁ and X₂ are NH.

In some embodiments, X₁ is O and X₂ is NH or X₁ is NH and X₂ is O.

In some embodiments of the compound of the invention, L is —C(O)—CH₂—OCH₂CH₂O—CH₂—C(O)—.

In some embodiments, L is —C(O)—CH₂—NHCH₂CH₂NH—CH₂—C(O)—, —C(O)—CH₂—NHCH₂CH₂O——CH₂—C(O)—, or —C(O)—CH₂—OCH₂CH₂NH—CH₂—C(O)—.

In some embodiments of the compound of the invention, T is a heterocycloalkyl moiety. In some embodiments, the heterocycloalkyl moiety is a piperazine moiety or a piperidine moiety.

In some embodiments of the compound of the invention, L is

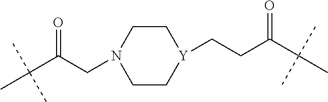

wherein Y is N or CH.

In some embodiments of the compound of the invention, L is -T-C(O)—(CH₂)ₘ—(CH₂)ₙ—C(O)—.

In some embodiments, T is an amino acid moiety. In some embodiments, the amino acid moiety is a lysine moiety or an arginine moiety.

In some embodiments of the compound of the invention, L is

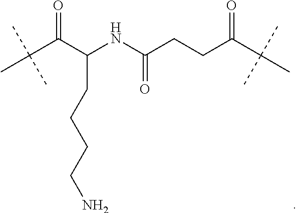

In some embodiments, L is

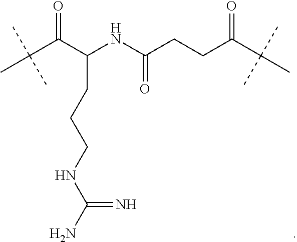

In some embodiments of the compound of the invention, L is -T-C(O)—CH₂—CH₂—C(O)—.

In some embodiments, T is a hydroxybenzoyl moiety.

In some embodiments of the compound of the invention, L is

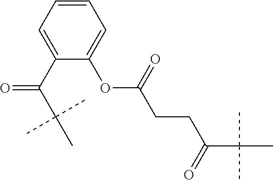

In some embodiments of the compound of the invention, L is —C(O)—CH₂—CH₂—C(O)-T-C(O)—CH₂—CH₂—C(O)—.

In some embodiments, T is an aminophenol moiety.

In some embodiments, L is

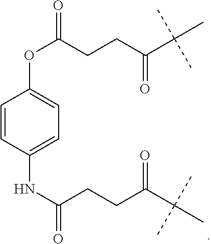

In some embodiments, G is NRₐR_{b}—C(NH)—.

In some embodiments, Rₐ is H and R_{b} is H.

In some embodiments, Rₐ is H and R_{b} is acetyl.

In some embodiments, G is a fenfluramine moiety.

In some embodiments, G is a serotonin moiety.

In some embodiments, the compound of formula (I) is represented by a compound of formula (II)

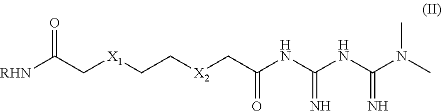

wherein RNH is a fenfluramine moiety or a serotonin moiety.

In some embodiments, X₁ and X₂ are O.

In some embodiments, X₁ and X₂ are NH.

In some embodiments, X₁ is O and X₂ is NH.

In some embodiments, X₁ is NH and X₂ is O.

In some embodiments, the compound of formula (I) is represented by a compound of formula (III)

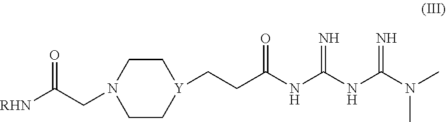

wherein RNH is a fenfluramine moiety or a serotonin moiety, and wherein Y is N or CH.

In some embodiments, Y is N.

In some embodiments, Y is CH.

In some embodiments, the compound of formula (1) is represented by a compound of formula (IV)

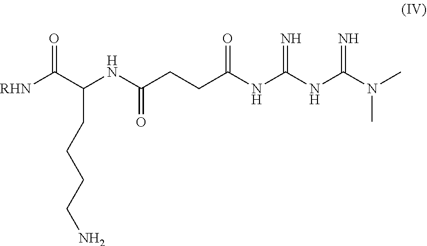

wherein RNH is a fenfluramine moiety or a serotonin moiety.

In some embodiments, the compound of formula (I) is represented by a compound of formula (V)

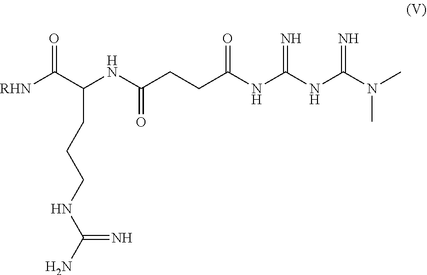

wherein RNH is a fenfluramine moiety or a serotonin moicty.

In some embodiments, the compound of the invention is

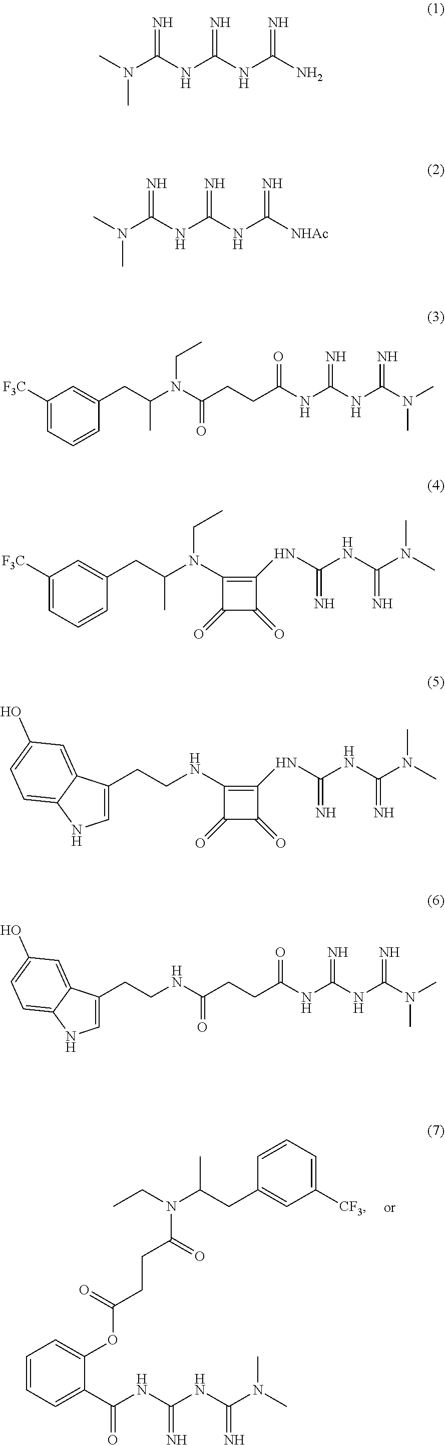

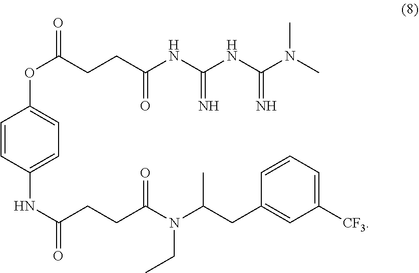 [SMILES: CCN(C(=O)CCC(=O)Nc1ccc(OC(=O)CCC(=O)NC(=N)NC(=N)N(C)C)cc1)C(C)Cc1cccc(C(F)(F)F)c1]

In some embodiments, the compound of the invention is compound (1)

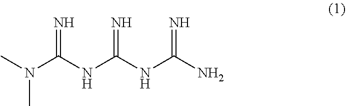

In some embodiments, the compound of the invention is compound (2)

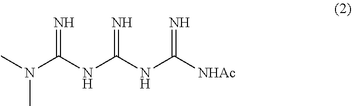

In some embodiments, the compound of the invention is compound (3)

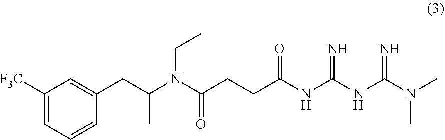 [SMILES: CCN(C(=O)CCC(=O)NC(=N)NC(=N)N(C)C)C(C)Cc1cccc(C(F)(F)F)c1]

In some embodiments, the compound of the invention is compound (4)

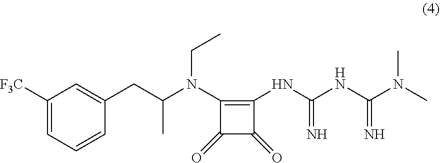 [SMILES: CCN(c1c(NC(=N)NC(=N)N(C)C)c(=O)c1=O)C(C)Cc1cccc(C(F)(F)F)c1]

In some embodiments, the compound of the invention is compound (5)

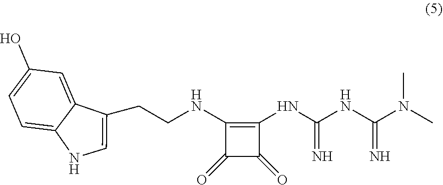 [SMILES: CN(C)C(=N)NC(=N)Nc1c(NCCc2c[nH]c3ccc(O)cc23)c(=O)c1=O]

In some embodiments, the compound of the invention is compound (6)

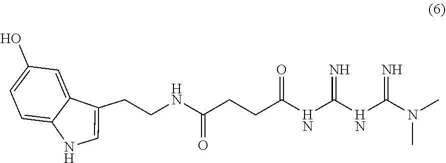 [SMILES: CN(C)C(=N)NC(=N)NC(=O)CCC(=O)NCCc1c[nH]c2ccc(O)cc12]

In some embodiments, the compound of the invention is compound (7)

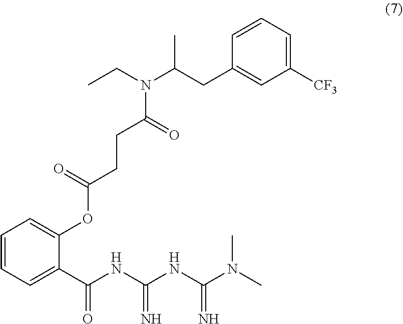 [SMILES: CCN(C(=O)CCC(=O)Oc1ccccc1C(=O)NC(=N)NC(=N)N(C)C)C(C)Cc1cccc(C(F)(F)F)c1]

In some embodiments, the compound of the invention is compound (8)

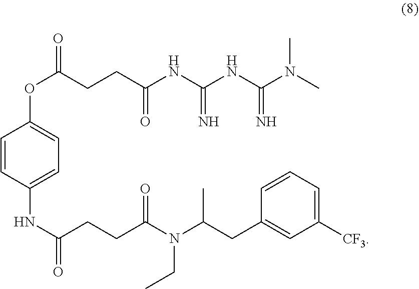 [SMILES: CCN(C(=O)CCC(=O)Nc1ccc(OC(=O)CCC(=O)NC(=N)NC(=N)N(C)C)cc1)C(C)Cc1cccc(C(F)(F)F)c1]

Unless defined otherwise, all technical and scientific terms used herein have the same meaning as commonly understood to one of ordinary skill in the art.

As used herein, in some embodiments, the term “alkyl” refers to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, the term “acyl” refers to hydrogen, alkyl, cycloalkyl, alkenyl, cycloalkenyl, heterocyclyl, aryl, and heteroaryl attached as a substituent through a carbonyl (C═O) group, such as formyl, acetyl, pivalolyl, benzoyl, and phenacetyl.

As used herein, in some embodiments, “heterocycloalkyl” refers to a non-aromatic heterocycle where one or more of the ring-forming atoms are a heteroatom such as an O, N, or S atom. Heterocycloalkyl groups can include mono- or polycyclic (e.g., having 2, 3 or 4 fused rings) ring systems as well as spirocycles. Example heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds.

As used herein, “serotonin” is as known in the art, which is also named as 3-(2-aminoethyl)-1H-indol-5-ol and has the following chemical structure:

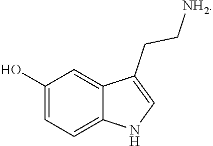 [SMILES: NCCc1c[nH]c2ccc(O)cc12]

As used herein, “dimethyl squarate” is as known in the art, which is named as 3,4-dimethoxy-3-cyclobutene-1,2-dione and has the following chemical structure:

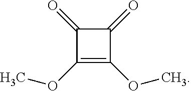

As used herein, “aspirin” is as known in the art, which is also named as 2-acetyloxybenzoic acid and has the following chemical structure:

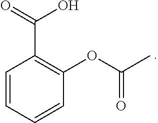

As used herein, “acetaminophen” is as known in the art, which is also named as 4-acetaminophenol or N-(4-hydroxyphenyl)acetamide and has the following chemical structure:

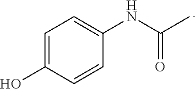

As used herein, “fenfluramine” is as known in the art, which is also named as N-ethyl-1-[3-(trifluoromethyl)phenyl]-2-propanamine and has the following chemical structure:

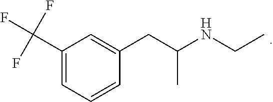 [SMILES: CCNC(C)Cc1cccc(C(F)(F)F)c1]

As used herein, “metformin” is as known in the art, which is also named as N,N-dimethylimidodicarbonimidic diamide and has the following chemical structure:

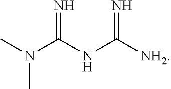

In each of the foregoing and each of the following embodiments, it is to be understood that the formulas also include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

As used herein, the term “solvate” refers to compounds that further include a stoichiometric or non-stoichiometric amount of a solvent bound by non-covalent intermolecular forces. If the solvent is water, the solvate is referred to as “hydrate.” Pharmaceutically acceptable solvates and hydrates are complexes that, for example, may include from 1 to about 100, or from 1 to about 10, or from one to about 2.3 or 4 molecules of water or a solvent. In some embodiments, the hydrate may be a channel hydrate. It should be understood that the term “compound” in this application covers the compound and solvates of the compound, as well as mixtures thereof.

In some embodiments, the term “hydrate” includes, but is not limited to, hemihydrate, monohydrate, dihydrate, trihydrate and the like.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds described herein may contain one or more asymmetric centers and may thus give rise to diastereomers and optical isomers. The present invention includes all such possible optical isomers, diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included.

In some embodiments, this invention encompasses the use of various optical isomers of the compound of the invention. It will be appreciated by those skilled in the art that the compounds of the present invention contain at least one chiral center. Accordingly, the compounds used in the methods of the present invention may exist in, and be isolated in, optically-active or racemic forms. Some compounds may also exhibit polymorphism. It is to be understood that the present invention encompasses any racemic, optically-active, polymorphic, or stereoisomeric form, or any combination thereof, which form possesses properties useful in the treatment of androgen-related conditions described herein. In some embodiments, the compounds of the inventio are optically pure. In other embodiments, the compounds of the invention are a racemic mixture. It is well known in the art how to prepare optically-active forms (for example, by resolution of the racemic form by recrystallization techniques, by synthesis from optically-active starting materials, by chiral synthesis, or by chromatographic separation using a chiral stationary phase).

During the course of the synthetic procedures used to prepare such compounds, or in using racemization or epimerization procedures known to those skilled in the art, the products of such procedures can be a mixture of stereoisomers.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes “pharmaceutically acceptable salts” of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compound of the invention include the conventional non-toxic salts of the parent compound formed, for example, from non-toxic inorganic or organic acids. The pharmaceutically acceptable salts of the compound of the invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In some embodiments, the solvent is a nonaqueous media like ether, ethyl acetate, ethanol, isopropanol, or acetonitrile.

The pharmaceutically acceptable salts of the compound of the invention can be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

Typically, a pharmaceutically acceptable salt form of a compound can be prepared in situ during the final isolation and purification of the compound, or separately by reacting the free base functionality with a suitable organic or inorganic acid.

Suitable acids for preparation of the pharmaceutically acceptable salts include, but are not limited to, inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods used in the art such as ion exchange.

Other pharmaceutically acceptable salts can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and quaternary ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The invention further includes derivatives of the compound of the invention. The term “derivatives” includes but is not limited to ether derivatives, acid derivatives, amide derivatives, ester derivatives and the like.

The invention further includes metabolites of the compound of the invention. The term “metabolite” means any substance produced from another substance by metabolism or a metabolic process.

The invention further includes pharmaceutical products of the compound of the invention. The term “pharmaceutical product” means a composition suitable for pharmaceutical use (pharmaceutical composition), as defined herein.

The invention further includes prodrugs of the compound of the invention. The term “prodrug” means a substance which can be converted in vivo into a biologically active agent by such reactions as hydrolysis, esterification, de-esterification, activation, salt formation and the like.

The present invention further provides a pharmaceutical composition comprising a compound of the invention, e.g., compound of formula (I) of the invention as described herein, or a pharmaceutically acceptable salt thereof, and a pharmaceutically acceptable carrier.

As used herein, “pharmaceutical composition” refers to a therapeutically effective amount of a compound of the present invention, or a pharmaceutically acceptable salt thereof, together with suitable diluents, preservatives, solubilizers, emulsifiers, adjuvant and/or carriers. Such compositions are liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

A “therapeutically effective amount” as used herein refers to that amount which provides a therapeutic effect for a given indication and administration regimen.

Numerous standard references are available that describe procedures for preparing various compositions or formulations suitable for administration of the compounds of the invention. Examples of methods of making formulations and preparations can be found in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage form are closely related to the therapeutic amounts of the compounds or compositions which are desirable and efficacious for the given treatment application.

The pharmaceutical compositions of the invention can be administered to a subject by any method known to a person skilled in the art. These methods include, but are not limited to, orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, by inhalation, rectally, or intratumorally. These methods include any means in which the composition can be delivered to tissue (e.g., needle or catheter). Alternatively, a topical administration may be desired for application to dermal, ocular, or mucosal surfaces. Another method of administration is via aspiration or aerosol formulation. The pharmaceutical compositions may be administered topically to body surfaces and are thus formulated in a form suitable for topical administration. Suitable topical formulations include gels, ointments, creams, lotions, drops and the like. For topical administrations, the compositions are prepared and applied as solutions, suspensions, or emulsions in a physiologically acceptable diluent with or without a pharmaceutical carrier.

Suitable dosage forms include, but are not limited to, oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. In some embodiments, the formulations are suitable for oral or topical administration.

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In preparing the compositions in oral dosage form, any of the usual pharmaceutical media may be employed. Thus, for liquid oral preparations, such as, suspensions, elixirs, and solutions, suitable carriers and additives include water, glycols, oils, alcohols, flavoring agents, preservatives, coloring agents, and the like. For solid oral preparations such as, powders, capsules, and tablets, suitable carriers and additives include starches, sugars, diluents, granulating agents, lubricants, binders, disintegrating agents, and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients may be included, such as ingredients that aid solubility or for preservation. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Methods of treatment using formulations suitable for oral administration may be presented as discrete units such as capsules, cachets, tablets, or lozenges, each containing a predetermined amount of the active ingredient. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration may comprise a sterile aqueous preparation of the active compound, which, in some embodiments, is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, compositions of this invention may further include one or more ingredient selected from diluents, buffers, flavoring agents, binders, disintegrants, surface active agents, thickeners, lubricants, preservatives (including antioxidants), and the like.

The formulations may be of immediate release, sustained release, delayed-onset release or any other release profile known to one skilled in the art.

For administration to mammals, and particularly humans, it is expected that the physician will determine the actual dosage and duration of treatment, which will be most suitable for an individual and can vary with the age, weight, genetics and/or response of the particular individual. In some embodiments, the subject is a mammal (e.g., a human or non-human mammal). In some embodiments, the subject is a human.

The methods of the invention comprise administration of a compound at a therapeutically effective amount. The therapeutically effective amount may include various dosages.

A dosage unit of the compounds used in the present invention may comprise a single compound or mixtures thereof with additional therapeutic agents. A “dose” or “dosage unit” or “unit dosage” of a compound of formula (I) of the invention as measured in milligrams refers to the milligrams of compound of formula (I) present in a preparation, regardless of the form of the preparation.

In some embodiments, a compound of the invention is administered at a dosage of 1-3000 mg per day. In some embodiments, a compound of the invention as described herein is administered at a dosage of 1-1000 mg per day. In some embodiments, a compound of the invention as described herein is administered at a dosage of 1-500 mg per day. In some embodiments, a compound of the invention as described herein is administered at a dosage of 10-500 mg per day. In some embodiments, a compound of the invention as described herein is administered at a dosage of 25-500 mg per day. In some embodiments, a compound of the invention as described herein is administered at a dosage of 50-500 mg per day. In some embodiments, a compound of the invention as described herein is administered at a dosage of 5-250 mg per day. In some embodiments, a compound of the invention as described herein is administered at a dosage of 10-250 mg per day. In some embodiments, a compound of the invention as described herein is administered at a dosage of 20-250 mg per day. In some embodiments, a compound of the invention as described herein is administered at a dosage of 25-250 mg per day. In some embodiments, a compound of the invention as described herein is administered at a dosage of 25-200 mg per day. In some embodiments, a compound of the invention as described herein is administered at a dosage of 25-150 mg per day. In some embodiments, a compound of the invention as described herein is administered at a dosage of 25-125 mg per day. In some embodiments, a compound of the invention as described herein is administered at a dosage of 25-100 mg per day.

In other embodiments, a compound of the invention is administered at a dose of 1-10 mg per day, 3-26 mg per day, 3-60 mg per day, 3-16 mg per day, 3-30 mg per day, 10-26 mg per day, 10-100 mg per day, 15-60 mg per day, 15-100 mg per day, 25-100 mg per day, 50-100 mg per day, 50-200 mg per day, 100-200 mg per day, 100-250 mg per day, 125-300 mg per day, 20-50 mg per day, 5-50 mg per day, 200-500 mg per day, 125-500 mg per day, 500-1000 mg per day, 200-1000 mg per day, 1000-2000 mg per day, 1000-3000 mg per day, 125-3000 mg per day, 2000-3000 mg per day, 300-1500 mg per day or 100-1000 mg per day.

The methods may comprise administering a compound at various dosages. For example, the compound may be administered per day at a dosage of 3 mg, 10 mg, 30 mg, 40 mg, 50 mg, 80 mg, 100 mg, 120 mg, 125 mg, 200 mg, 250 mg, 300 mg, 450 mg, 500 mg, 600 mg, 900 mg, 1000 mg, 1500 mg, 2000 mg, 2500 mg or 3000 mg.

Alternatively, the compound may be administered at a dosage of 0.1 mg/kg/day. The compound may be administered at a dosage between 0.2 to 30 mg/kg/day, or 0.2 mg/kg/day, 0.3 mg/kg/day, 1 mg/kg/day, 3 mg/kg/day, 5 mg/kg/day, 10 mg/kg/day, 20 mg/kg/day, 30 mg/kg/day, 50 mg/kg/day or 100 mg/kg/day.

In some embodiments, a dosage unit can be prepared for oral dosage forms, such as tablets, capsules, pills, powders, liquid suspensions, and granules.

The invention further provides a pharmaceutical composition comprising a compound of formula (I) as described herein, or a pharmaceutically acceptable salt thereof, for use treating diabetes in a subject in need thereof. In some embodiments, the diabetes is type 2 diabetes. In some embodiments, the subject is obese. In other embodiments, the subject is overweight.

The invention further provides a pharmaceutical composition comprising a compound of formula (I) as described herein, or a pharmaceutically acceptable salt thereof, for use for treating type 2 diabetes. In some embodiments, the subject is obese. In other embodiments, the subject is overweight.

The invention further provides a pharmaceutical composition comprising a compound of formula (I) as described herein, or a pharmaceutically acceptable salt thereof, for use for treating obesity or inducing weight reduction in a subject in need thereof. In some embodiments, the subject has diabetes. In some embodiments, the subject has type 2 diabetes.

In some embodiments, the pharmaceutical composition of the invention is in the form of a capsule, a tablet, or a liquid suspension. In other embodiments, the pharmaceutical composition of the invention is in an oral dosage unit form.

The exact dose and regimen of administration of the composition will necessarily be dependent upon the therapeutic or nutritional effect to be achieved and may vary with the particular formula, the route of administration, and the age and condition of the individual subject to whom the composition is to be administered.

In some embodiments, a pharmaceutical composition is prepared for once daily administration. In other embodiments, a pharmaceutical composition is prepared for more than once daily administration, for example, twice daily, three times daily, four times daily, etc.

The invention further provides a method for treating diabetes in a subject in need thereof, comprising administering to the subject a compound of formula (I) as described herein, or a pharmaceutically acceptable salt thereof. In some embodiments, the diabetes is type 2 diabetes. In some embodiments, the subject is obese. In other embodiments, the subject is overweight.

The invention further provides a method for treating type 2 diabetes in a subject in need thereof, comprising administering to the subject a compound of formula (I) as described herein, or a pharmaceutically acceptable salt thereof. In some embodiments, the subject is obese. In other embodiments, the subject is overweight.

The invention further provides a method for treating obesity or inducing weight reduction in a subject in need thereof, comprising administering to the subject a compound of formula (I) as described herein, or a pharmaceutically acceptable salt thereof. In some embodiments, the subject has diabetes. In some embodiments, the subject has type 2 diabetes. In some embodiments, the subject is obese. In other embodiments, the subject is overweight.

In some embodiments of the methods of the invention, the compound is represented by formula (I)

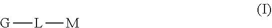

wherein
G is NRₐR_{b}—C(NH)—, a fenfluramine moiety, or a serotonin moiety;
M is —NH—C(NH)—NH—C(NH)—N(CH₃)₂;
L is a linker selected from the group consisting of a bond, —C(O)—(CH₂)ₘ-T-(CH₂)ₙ—C(O)—, -T-C(O)—CH₂—CH₂—C(O)—, —C(O)—CH₂—CH₂—C(O)-T-, —C(O)—CH₂—CH₂—C(O)-T-C(O)—CH₂—CH₂—C(O)—, and a 3-cyclobutene-1,2-dione moiety;
T is a bond, —X₁—CH₂—CH₂—X₂—, an amino acid moiety, a heterocycloalkyl moiety, a hydroxybenzoyl moiety, or an aminophenol moiety;
X₁ and X₂ are each O or NR_{c};
Rₐ, R_{b}, and R_{c} are each independently H, an alkyl group, or an acyl group, and
m and n are each 1, 2, or 3,
   or a pharmaceutically acceptable salt thereof.

In some embodiments, L is a bond.

In some embodiments, L is —C(O)—CH₂—CH₂—C(O)—.

In some embodiments, L is a 3-cyclobutene-1,2-dione moiety.

In some embodiments, L is —C(O)—(CH₂)ₘ-T-(CH₂)ₙ—C(O)—.

In some embodiments, m is 1 and n is 1.

In some embodiments of the methods of the invention, L is

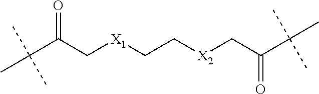

In some embodiments, X₁ and X₂ are O.

In some embodiments, X₁ and X₂ are NH.

In some embodiments, X₁ is O and X₂ is NH or X₁ is NH and X₂ is O.

In some embodiments of the methods of the invention, L is —C(O)—CH₂—OCH₂CH₂O—CH₂—C(O)—.

In some embodiments, L is —C(O)—CH₂—NHCH₂CH₂NH—CH₂—C(O)—, —C(O)—CH₂—NHCH₂CH₂O—CH₂—C(O)—, or —C(O)—CH₂—OCH₂CH₂NH—CH₂—C(O)—.

In some embodiments of the methods of the invention, T is a heterocycloalkyl moiety. In some embodiments, the heterocycloalkyl moiety is a piperazine moiety or a piperidine moiety.

In some embodiments of the methods of the invention, L is

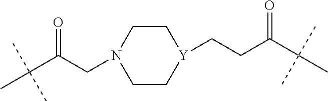

wherein Y is N or CH.

In some embodiments of the methods of the invention, L is -T-C(O)—(CH₂)ₘ—(CH₂)ₙ—C(O)—.

In some embodiments, T is an amino acid moiety. In some embodiments, the amino acid moiety is a lysine moiety or an arginine moiety.

In some embodiments of the methods of the invention, L is

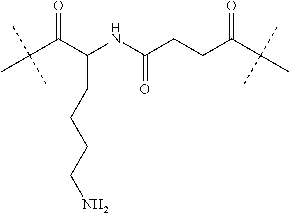

In some embodiments, L is

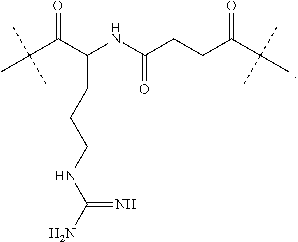

In some embodiments of the methods of the invention, L is -T-C(O)—CH₂-CH₂—C(O)—.

In some embodiments, T is a hydroxybenzoyl moiety.

In some embodiments of the methods of the invention, L is

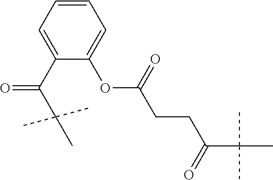

In some embodiments of the methods of the invention, L is —C(O)—CH₂-CH₂—C(O)—T—C(O)—CH₂—CH₂—C(O)—.

In some embodiments, T is an aminophenol moiety.

In some embodiments, L is

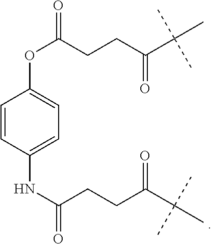

In some embodiments, G is NRₐR_{b}—C(NH)—.

In some embodiments, Rₐ is H and R_{b} is H.

In some embodiments, Rₐ is H and R_{b} is acetyl.

In some embodiments, G is a fenfluramine moiety.

In some embodiments, G is a serotonin moiety.

In some embodiments of the methods of the invention, the compound of formula (I) is represented by a compound of formula (II)

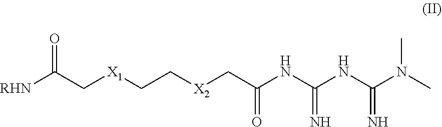

wherein RNH is a fenfluramine moiety or a serotonin moiety.

In some embodiments, X₁ and X₂ are O.

In some embodiments, X₁ and X₂ are NH.

In some embodiments, X₁ is O and X₂ is NH.

In some embodiments, X₁ is NH and X₂ is O.

In some embodiments of the methods of the invention, the compound of formula (I) is represented by a compound of formula (III)

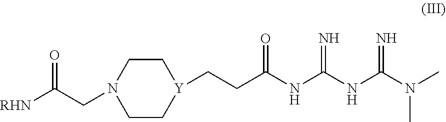

wherein RNH is a fenfluramine moiety or a serotonin moiety, and wherein Y is N or CH.

In some embodiments, Y is N.

In some embodiments, Y is CH.

In some embodiments of the methods of the invention, the compound of formula (I) is represented by a compound of formula (IV)

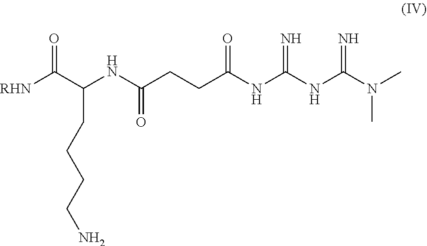

wherein RNH is a fenfluramine moiety or a serotonin moiety.

In some embodiments of the methods of the invention, the compound of formula (I) is represented by a compound of formula (V)

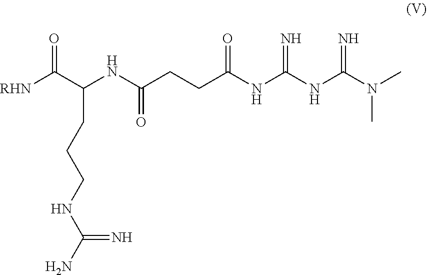

wherein RNH is a fenfluramine moiety or a serotonin moiety.

In some embodiments of the methods of the invention, the compound of the invention is

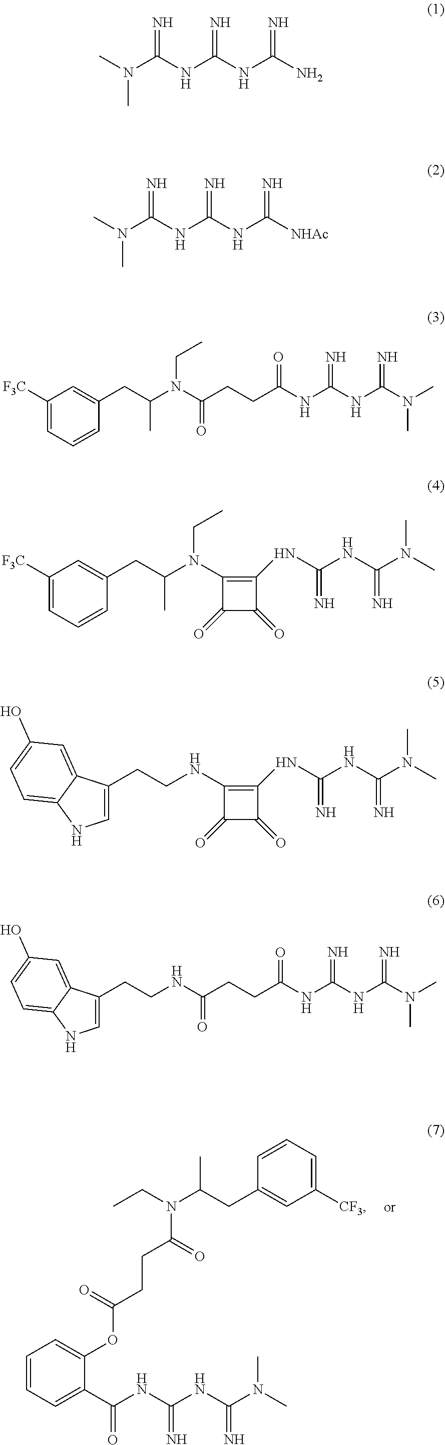

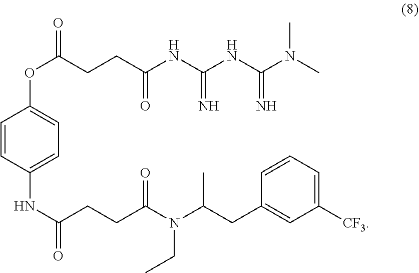 [SMILES: CCN(C(=O)CCC(=O)Nc1ccc(OC(=O)CCC(=O)NC(=N)NC(=N)N(C)C)cc1)C(C)Cc1cccc(C(F)(F)F)c1]

In some embodiments of the methods of the invention, the compound is compound (1)

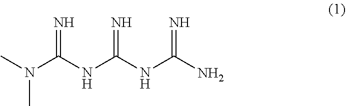

In some embodiments of the methods of the invention, the compound is compound (2)

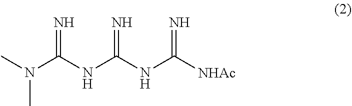

In some embodiments of the methods of the invention, the compound is compound (3)

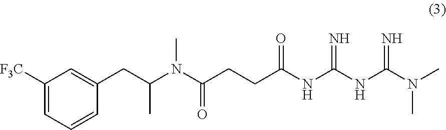 [SMILES: CC(Cc1cccc(C(F)(F)F)c1)N(C)C(=O)CCC(=O)NC(=N)NC(=N)N(C)C]

In some embodiments of the methods of the invention, the compound is compound (4)

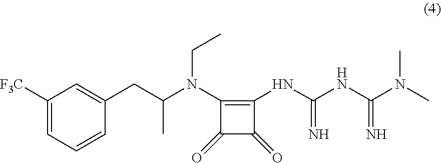 [SMILES: CCN(c1c(NC(=N)NC(=N)N(C)C)c(=O)c1=O)C(C)Cc1cccc(C(F)(F)F)c1]

In some embodiments of the methods of the invention, the compound is compound (5)

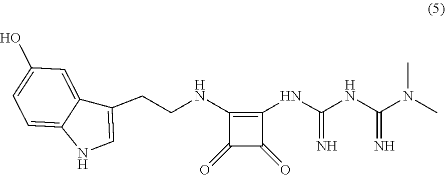 [SMILES: CN(C)C(=N)NC(=N)Nc1c(NCCc2c[nH]c3ccc(O)cc23)c(=O)c1=O]

In some embodiments of the methods of the invention, the compound is compound (6)

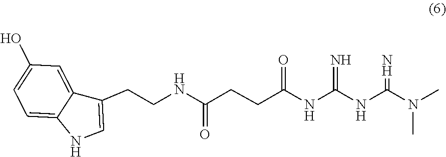 [SMILES: CN(C)C(=N)NC(=N)NC(=O)CCC(=O)NCCc1c[nH]c2ccc(O)cc12]

In some embodiments of the methods of the invention, the compound is compound (7)

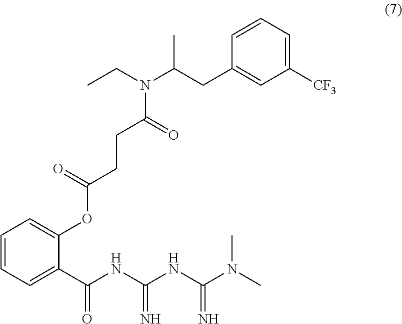 [SMILES: CCN(C(=O)CCC(=O)Oc1ccccc1C(=O)NC(=N)NC(=N)N(C)C)C(C)Cc1cccc(C(F)(F)F)c1]

In some embodiments of the methods of the invention, the compound is compound (8)

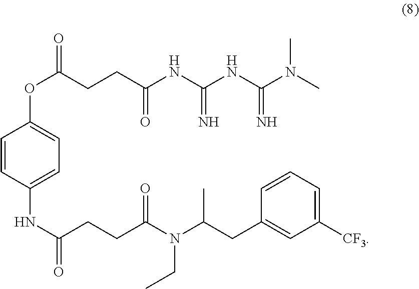 [SMILES: CCN(C(=O)CCC(=O)Nc1ccc(OC(=O)CCC(=O)NC(=N)NC(=N)N(C)C)cc1)C(C)Cc1cccc(C(F)(F)F)c1]

In some embodiments, the described methods of treatment can additionally include administering to the subject one or more additional therapeutic agents for treating type 2 diabetes. Such other therapeutic agents may be administered, by a route and in an amount commonly used therefore, simultaneously or sequentially with a compound or composition of the present invention. In certain embodiments, the compounds of the invention as described herein can be combined with one or more therapeutic agents for treating type 2 diabetes, for example, SGLT2 inhibitors, GLP-1 and GLP-1+agonists (especially derivatized with metformin type), targeting PPARγ GLUT-4 such as thiazolidinediones (pioglitazone and rosiglitazone).

The present invention further provides a combination therapy. The term “combination therapy” means the administration of two or more therapeutic agents to treat a therapeutic disorder described in the present invention. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agents in a sequential manner. In either case, the treatment regimen will provide beneficial effects of the drug combination in treating the type 2 diabetes described herein.

As used herein, the singular forms “a”, “an” and “the” include plural referents unless the content clearly dictates otherwise.

The terms “subject” and “patient” are used interchangeably herein when referencing, for example, a mammalian subject, such as a human patient. In some embodiments, the subject in the method of the invention is a human patient.

It is to be understood that the phraseology or terminology employed herein, and not otherwise defined, is for the purpose of description only and not of limitation. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. Further, reference to values stated in ranges includes each and every value within that range. Certain features of the disclosed compositions and methods which are described herein in the context of separate aspects may also be provided in combination in a single aspect. Alternatively, various features of the disclosed compositions and methods that are, for brevity, described in the context of a single aspect, may also be provided separately or in any subcombination.

The invention further provides processes for the preparation of compounds of formula (I). For example, Compounds of formula (I) can be prepared using methods known in the art, including, for example, by referring to the following schemes.

The following examples are presented in order to more fully illustrate the embodiments of the invention. They should in no way, however, be construed as limiting the broad scope of the invention.

### EXAMPLE I: Preparation of Compounds of Invention

The invention further provides the processes for the preparation of compounds of the present invention. Schemes 1 and 2 provide processes for the preparation of the compounds of the invention.

### Example 1: Synthesis of N-acetyl N,N-dimethyl triguanidine

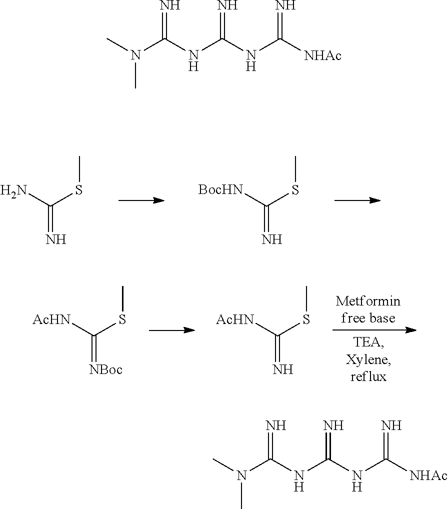

N-acetyl-S-methylisothiourea was prepared following the reported procedure starting from commercially available S-methylisothiouronium hemisulfate [Angew Chemie Int. ed. 2016, 55(4) 1540-1543] in three steps with modification in the deprotection of Boc group. N-(tert-butoxycarbonyl)-S-methylisothiourea

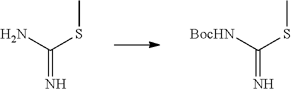

A mixture of S-methylisothiuronium hemisulfate (2.8 g, 10 mmol) solution in bicarbonate (sat'd, 30 ml) and a solution of di(tert-butyl) dicarbonate (4.4 g, 10 mmol) in CH₂Cl₂ (30 ml) was vigorously stirred overnight at 25° C. Layers were separated and the aqueous phase was extracted with methylene chloride (2×15 ml). The organic phases were combined, dried over Na₂SO₄ and the solvent removed under reduced pressure. After flash chromatography (hexane/EtOAc 4:1), 1.6 g (42%) the title compound was obtained as a colorless, viscous solid.

### N-Acetyl-N′-tert-butyloxycarbonyl-S-methylisothiourea

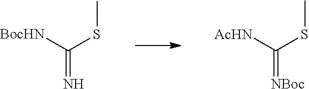

To a solution of N-(tert-butoxycarbonyl)-S-methylisothiourea (1.3 g, 6.84 mmol) in methylene chloride (30 mL) acetic anhydride (0.75 ml, 7.5 mmol) was added followed by TEA (1.5 mL, 10.9 mmol). The mixture was stirred for 3 hours at room temperature. The disappearance of starting material was indicated by TLC. The resulting mixture was diluted with methylene chloride (50 mL), washed with brine, dried over Na₂SO₄, filtered and concentrated. The crude product was found to be pure by TLC and was used in the next step without purification, 1.2 g yield.

### N-Acetyl-S-methylisothiourea

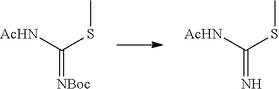

The N-acetyl-N′-tert-butyloxycarbonyl-S-methylisothiourea (1.54 g, 6.63 mmol) was treated with TFA (10 mL). The resulting mixture was stirred for 30 minutes at room temperature, evaporated to dryness and co-evaporated with acetonitrile twice. The residue was stirred with excess of EtOAc and the product precipitated as a white solid, which was filtered, and dried. This crude product was used without further purification.

### N-Acetyl N,N-dimethyl triguanidine

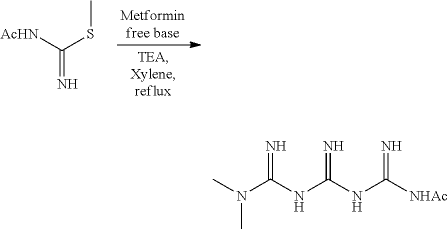

A mixture of N-acetyl-S-methylisothiourea (1.4 g, 10.6 mmol), metformin free base (1.8 g, 13.9 mmol) [Synthesis 2008, 22, 3619-3624] and triethylamine (1.8 ml) in a mixture of DCM: xylenes (1:1, 20 mL) was heated slowly in an oil bath to 100° C. for an hour and later to 145-150° C. for 5 h. Once cooled to 25° C., the reaction mixture was concentrated to remove solvent. The crude reaction mixture was purified by flash column chromatography using methylene chloride and MeOH (0-10%). Fractions with the same Rf were collected and concentrated to give the product as white solid (700 mg, 31%). ¹H NMR (400 MHz, DMSO-d6) δ2.07 (s 3H), 3.02 (s, 6H).

### Example 2: Synthesis of Fenfluramine Linked Metformin

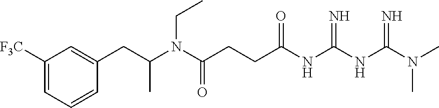 [SMILES: CCN(C(=O)CCC(=O)NC(=N)NC(=N)N(C)C)C(C)Cc1cccc(C(F)(F)F)c1]

### Preparation of Racemic Fenfluramine

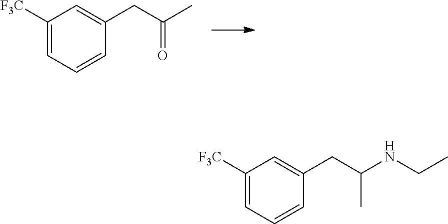 [SMILES: CC(=O)Cc1cccc(C(F)(F)F)c1 ; CCNC(C)Cc1cccc(C(F)(F)F)c1]

Reductive amination of 3-fluorophenylacetone with ethylamine was carried out following a reported procedure [Synlett 2012, 23, 2176] in the presence of DIPEA and MgSO₄ using sodium triacetoxyborohydride. ¹H NMR (400 MHZ, CDCl₃) δ1.2 (m, 5 H, 2CH₃), 2.6-3.0 (m, 5 H), 7.65 (m, 4 H).

### Methyl 4-[(1-methyl-3-(trifluoromethyl)phenylethyl)amino]-4-oxo-butanoate

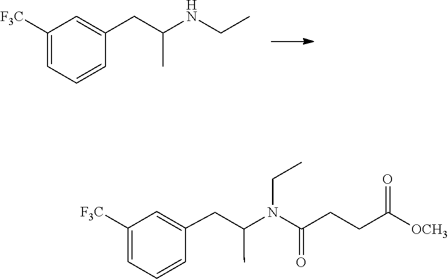 [SMILES: CCNC(C)Cc1cccc(C(F)(F)F)c1 ; CCN(C(=O)CCC(=O)OC)C(C)Cc1cccc(C(F)(F)F)c1]

To an ice-cooled solution of fenfluramine (4.7 g, 17.5 mmol) in methylene chloride (30 mL), triethyl amine (4.2 mL) was added followed by slow addition of methyl 4-chloro-4-oxobutyate (3.66 g, 24.35 mmol) in methylene chloride (10 mL). To the cloudy reaction mixture DCM (20 ml) was added to obtain a clear solution. The reaction mixture was stirred at 0-5° C. for one hour and then stirred for at 25° C. for 4 h. After the completion of reaction, as indicated by TLC (methylene chloride), water was added to the reaction mixture, layers were separated and the aqueous phase was extracted with methylene chloride (2×10 mL). Combined organic layers were washed with brine, dried (Na₂SO₄), and filtered. After solvent removal, the crude mixture was purified by column chromatography (silica gel) using methylene chloride which afforded product an oil (5.6 g, 84%).

### Hydrolysis of methyl ester

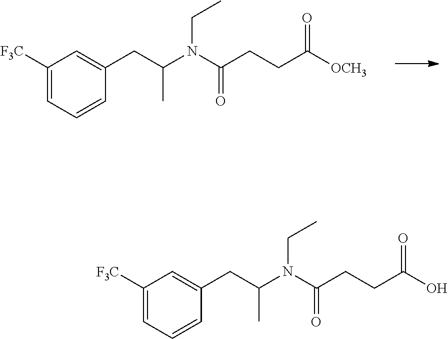 [SMILES: CCN(C(=O)CCC(=O)OC)C(C)Cc1cccc(C(F)(F)F)c1 ; CCN(C(=O)CCC(=O)O)C(C)Cc1cccc(C(F)(F)F)c1]

LiOH monohydrate (1.36 g, 32.41 mmol) was added to a solution of the above methyl ester (5.6 g, 14.66 mmol) in methanol-water (9:1, 50 mL) and the mixture was stirred overnight. Hydrolysis was monitored by TLC indicated by the disappearance of the ester. At the end of reaction, the mixture was concentrated, diluted with water (50 mL), and the aqueous layer was extracted with EtOAc (10 mL) to remove any trace amounts of unhydrolyzed ester. The aqueous layer was cooled and acidified with conc. HCl to acidic pH (˜2). Solid NaCl (20% by vol) was added to the solution and the mixture was extracted twice with methylene chloride (2×50 mL). The mixture was dried (Na₂SO₄), filtered, concentrated and the resulting acid (4.6 g, 83%) was used without any further purification.

### Coupling of fenfluramine with metformin

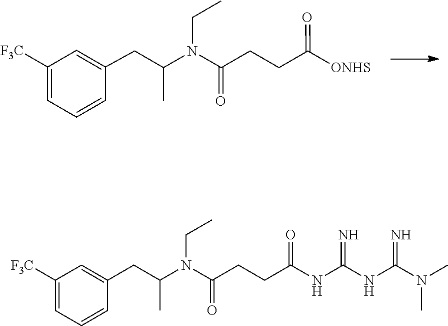

N-Hydroxysuccinimide (0.58 g, 5.1 mmol) was added to the above fenfluramine-carboxylic acid derivative (1.6 g, 4.35 mmol) in methylene chloride (15 mL) at 5-0° C. DCC (1.43 g, 4.83 mmol) was added in portions over 10 mins period. The resultant suspension was stirred 16 h and solids were removed under filtration. The filtrate was concentrated to give the active NHS ester, which was used without further purification.

The above crude NHS ester was dissolved in THF (10 mL) and metformin free base (0.62 g, 4.83 mmol) was added followed by the addition of TEA (0.7 mL, 4.83 mmol). Addition of few drops of water resulted in a clear solution which was stirred at 25° C.overnight. The reaction mixture was concentrated and purified by column chromatography using methylene chloride-MeOH (0-10% gradient). Appropriate fractions were combined, concentrated and dried under high vacuum. Yield 300 mg (15%); ¹H NMR (400 MHZ, CDCl₃) δ1.2 (m, 5 H, 2CH₃), 2.6-3.0 (m, 5 H), 3.16 (s 3H), (7.65 (m, 4 H).

### Example 3: Synthesis of N,N-dimethyl triguanidine

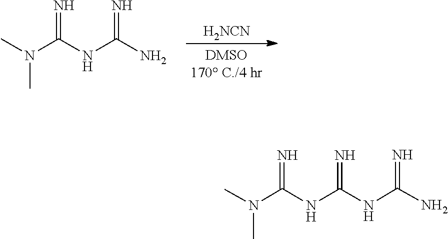

N,N-Dimethyl triguanidine was prepared using a modified method of KN Nandi. [Nandi K N, Phillips M A. Preparation of triguanide and of certain alkyl substituted triguanides. Chemistry & Industry (London, United Kingdom) 1958, 719.] Metformin HCl (10 g, 77.5 mmol) was heated at 170° with cyanamide (3.2 g, 77.5 mmol) in DMSO (20 mL) for 4 hrs. DMSO was removed by evaporation using an air stream and then water (10 mL) was added to dissolve the solid. The mixture was purified by Combi flash: 100 g C18 HP column (RediSep gold, Teledyne Isco) using a 0 to 10% gradient of acetonitrile in 0.1 M ammonium formate. After water evaporation, the ammonium formate was removed from the product by Combi flash using a 0 to 10% gradient of acetonitrile. The solution was acidified using 10% HCl and solvent was evaporated. Yield: 10%. 1H NMR (400 MHZ, DMSO-d₆) δ3.02 (s, 6H). HPLC (C-18 Waters Xbridge column, 0-10% acetonitrile in 0.1 M ammonium formate), retention time 4 min. No metformin was detected, retention time 3.5 min.

### Example 4: Serotonin linked metformin

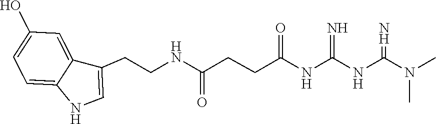 [SMILES: CN(C)C(=N)NC(=N)NC(=O)CCC(=O)NCCc1c[nH]c2ccc(O)cc12]

### Methyl-4-oxo-butanoate 5-hydroxytryptamine derivative

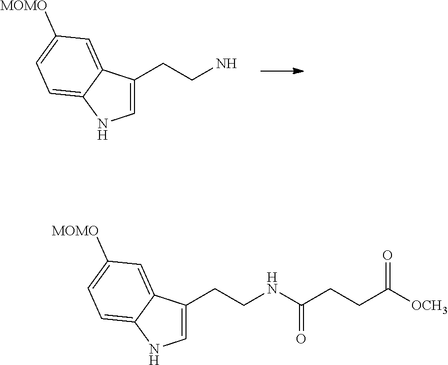

Using the same procedure as described for fenfluramine linked metformin, an ice-cooled solution of methoxymethyl ether-serotonin in methylene chloride (30 mL), triethyl amine (4.2 mL) is added followed by slow addition of methyl 4-chloro-4-oxobutyate (3.66 g, 24.35 mmol) in methylene chloride (10 ml). To the cloudy reaction mixture DCM (20 ml) was added to obtain a clear solution. The reaction mixture is stirred at 0-5° C. for one hour and then stirred for at 25° C. for 4 h. After the completion of reaction, as indicated by TLC (methylene chloride), water is added to the reaction mixture, layers are separated and the aqueous phase is extracted with methylene chloride (2×10 mL). Combined organic layers are washed with brine, dried (Na₂SO₄), and filtered. After solvent removal, the crude mixture is purified by column chromatography (silica gel) using methylene chloride which affords the product as an oil.

### Hydrolysis of Methyl Ester

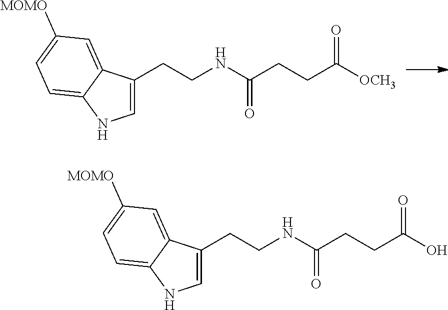

LiOH monohydrate (1.36 g, 32.41 mmol)) is added to a solution of the above methyl ester (5.6 g, 14.66 mmol) in methanol-water (9:1, 50 mL) and the mixture is stirred overnight. Hydrolysis is monitored by TLC indicated by the disappearance of the ester. At the end of reaction, the mixture is concentrated, diluted with water (50 mL), and the aqueous layer is extracted with EtOAc (10 mL) to remove any trace amounts of unhydrolyzed ester. The aqueous layer is ice-cooled and acidified with 10% HCl to acidic pH (˜2). Solid NaCl (20%) was added to the solution and the mixture was extracted twice with methylene chloride (2×50 mL). The mixture was dried (Na₂SO₄), filtered, concentrated and the resulting acid is used without any further purification.

### Coupling of Serotonin with Metformin

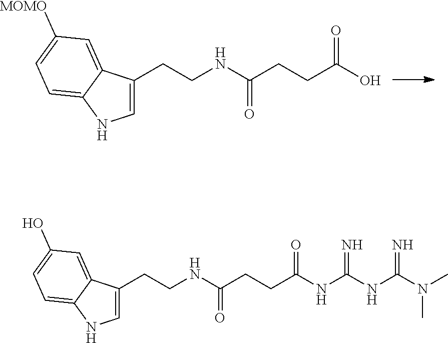

N-Hydroxysuccinimide is added to the above serotonin-carboxylic acid derivative in methylene chloride (15 mL) at 5-0 ° C. DCC is added in portions over 10 mins period. The resultant suspension is stirred for 16 h and solids are removed under filtration. The filtrate is concentrated to give the active NHS ester, which was used without further purification.

The above crude NHS ester is dissolved in THF (10 mL) and metformin free base is added followed by the addition of TEA. Addition of few drops of water results in a clear solution which is stirred at 25° C.stirred overnight. MOM-deprotection is achieved by using TFA in methylene chloride. The reaction mixture is quenched with water and the methylene chloride layer is separated, dried and concentrated. The product is purified by column chromatography using methylene chloride-MeOH (0-20% gradient).

### Example 5: Squaramide Linkage Coupling of Serotonin with Metformin

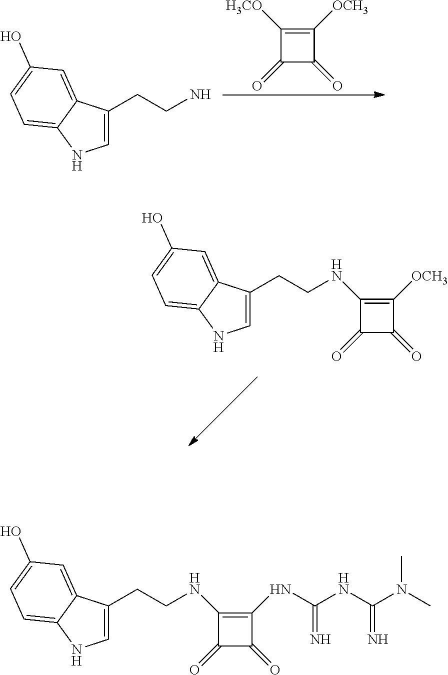 [SMILES: NCCc1c[nH]c2ccc(O)cc12 ; COc1c(OC)c(=O)c1=O ; COc1c(NCCc2c[nH]c3ccc(O)cc23)c(=O)c1=O ; CN(C)C(=N)NC(=N)Nc1c(NCCc2c[nH]c3ccc(O)cc23)c(=O)c1=O]

Squaramides are easily prepared in a two-step process. Dimethyl squarate reacts with 5-hydroxy-tryptamine in DCM or MeOH under cold conditions. Next, coupling with metformin in ethanol/THF under reflux results in the disubstituted squaramide, which usually precipitates out of solution.

### Example 6: Ethylene Glycol Linkage Coupling of Serotonin with Metformin

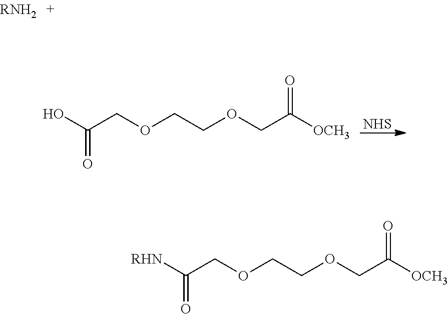

N-Hydroxysuccinimide is added to ethylene glycol carboxylic acid derivative in methylene chloride (15 mL) at 5-0° C. DCC is added in portions over 10 mins period. The resultant suspension is stirred for 16 h and solids are removed under filtration. The filtrate is concentrated to give the active NHS ester, which is used without further purification. The crude NHS ester is dissolved in THF (10 mL) and serotonin free base is added followed by the addition of TEA. Addition of few drops of water results in a clear solution which is stirred at 25° C. stirred overnight.

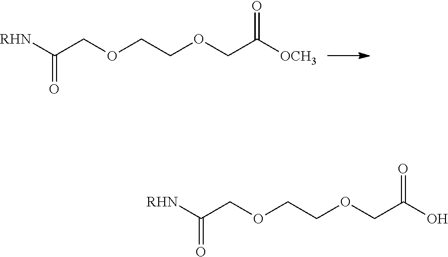

LiOH monohydrate is added to a solution of the above methyl ester in methanol-water (9:1, 50 mL) and the mixture is stirred overnight. Hydrolysis is monitored by TLC indicated by the disappearance of the ester. At the end of reaction, the mixture is concentrated, diluted with water (50 mL), and the aqueous layer is extracted with EtOAc (10 mL) to remove any trace amounts of unhydrolyzed ester. The aqueous layer is cooled and acidified with conc. HCl to acidic pH (˜2). Solid NaCl (20% by vol) is added to the solution and the mixture is extracted twice with methylene chloride (2×50 mL). The mixture is dried (Na₂SO₄), filtered, concentrated and the resulting acid is used without any further purification.

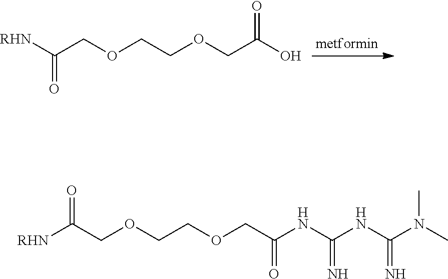

N-Hydroxysuccinimide is added to the above serotonin-carboxylic acid derivative in methylene chloride (15 mL) at 5-0° C. DCC is added in portions over 10 mins period. The resultant suspension is stirred for 16 h and solids are removed under filtration. The filtrate is concentrated to give the active NHS ester, which is used without further purification. The above crude NHS ester is dissolved in THF (10 mL) and metformin free base is added followed by the addition of TEA. Addition of few drops of water results in a clear solution which is stirred at 25° C. stirred overnight. The product is purified by column chromatography using methylene chloride-MeOH (0-20% gradient).

### Example 7: Piperazine Linkage Coupling of Serotonin with Metformin

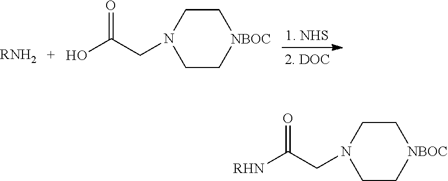

This synthesis can be done by using either DCC/NHS or HATU.

N-Hydroxysuccinimide is added to Boc-protected piperazine carboxylic acid derivative in methylene chloride (15 mL) at 5-0° C. DCC is added in portions over 10 mins period. The resultant suspension is stirred for 16 h and solids are removed under filtration. The filtrate is concentrated to give the active NHS ester, which is used without further purification. The crude NHS ester is dissolved in THF (10 mL) and serotonin free base is added followed by the addition of TEA. Addition of few drops of water results in a clear solution which is stirred at 25° C. stirred overnight.

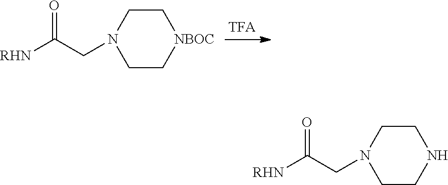

BOC deprotection is achieved by using TFA in methylene chloride. The reaction mixture is quenched with water and the methylene chloride layer is separated, dried and concentrated.

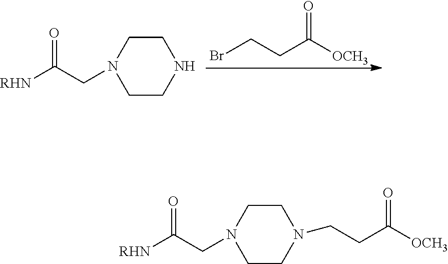

Alkylation of the piperazine derivative is achieved using methyl 3-bromopropionate ester (1 mol eq.) and N,N-diisopropylethylamine (1.5 mol eq.) in anhydrous DMF at room temperature under nitrogen until completion of reaction.

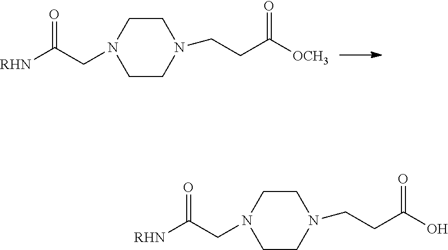

LiOH monohydrate is added to a solution of the above methyl ester in methanol-water (9:1, 50 mL) and the mixture is stirred overnight. Hydrolysis is monitored by TLC indicated by the disappearance of the ester. At the end of reaction, the mixture is concentrated, diluted with water (50 mL), and the aqueous layer is extracted with EtOAc (10 mL) to remove any trace amounts of unhydrolyzed ester. The aqueous layer is cooled and acidified with conc. HCl to acidic pH (˜2). Solid NaCl (20% by vol) is added to the solution and the mixture was extracted twice with methylene chloride (2×50 mL). The mixture is dried (Na₂SO₄), filtered, concentrated and the resulting acid is used without any further purification.

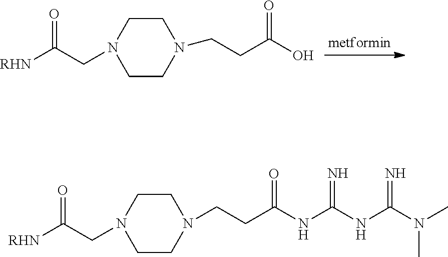

N-Hydroxysuccinimide is added to the above serotonin-carboxylic acid derivative in methylene chloride (15 mL) at 5-0° C. DCC is added in portions over 10 mins period. The resultant suspension is stirred for 16 h and solids are removed under filtration. The filtrate is concentrated to give the active NHS ester, which was used without further purification. The above crude NHS ester is dissolved in THF (10 mL) and metformin free base is added followed by the addition of TEA. Addition of few drops of water results in a clear solution which was stirred at 25° C. stirred overnight. The reaction mixture is quenched with water and the methylene chloride layer is separated, dried and concentrated. The product is purified by column chromatography using methylene chloride-MeOH (0-20% gradient).

### Example 8: Lysine Linkage Coupling of Serotonin with Metformin

Peptide bonds used in lysine or arginine linker function are hydrolyzed by digestive enzymes trypsin and chymotrypsin

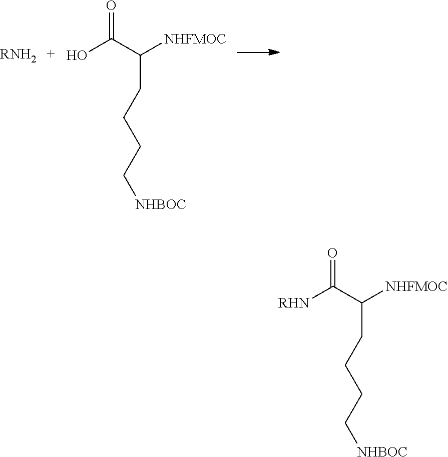

N-Hydroxysuccinimide is added to the serotonin-carboxylic acid derivative in methylene chloride (15 mL) at 5-0° C. DCC is added in portions over 10 mins period. The resultant suspension is stirred for 16 h and solids are removed under filtration. The filtrate is concentrated to give the active NHS ester, which is used without further purification. The above crude NHS ester is dissolved in THF (10 mL) and serotonin free base is added followed by the addition of TEA. Addition of few drops of water results in a clear solution which is stirred at 25° C. stirred overnight. The product is purified by column chromatography using methylene chloride-MeOH (0-20% gradient).

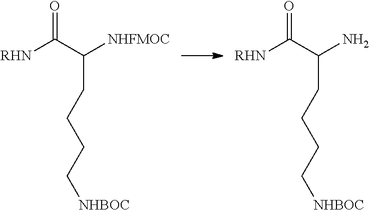

FMOC deprotection of the serotoin-lysine derivative is achieved by using 4% piperidine in DMF at room temperature.

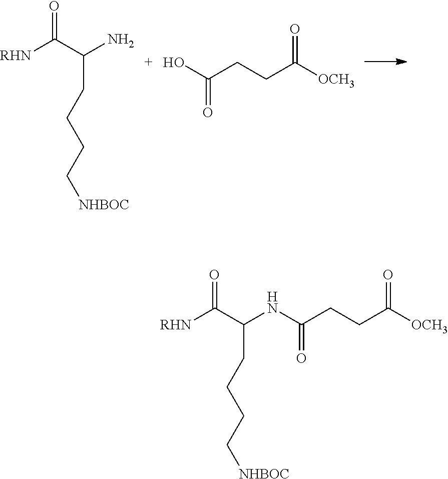

N-Hydroxysuccinimide is added to the above carboxylic acid ester derivative in methylene chloride (15 mL) at 5-0° C. DCC is added in portions over 10 mins period. The resultant suspension is stirred for 16 h and solids are removed under filtration. The filtrate is concentrated to give the active NHS ester, which was used without further purification. The above crude NHS ester is dissolved in THF (10 mL) and lysine-serotoin derivative free base is added followed by the addition of TEA. Addition of few drops of water resulted in a clear solution which was stirred at 25° C. stirred overnight. The product is purified by column chromatography using methylene chloride-MeOH (0-20% gradient). Note: The ester can also be directly used for reaction with metformin in the presence of boric acid or NH₄Cl.

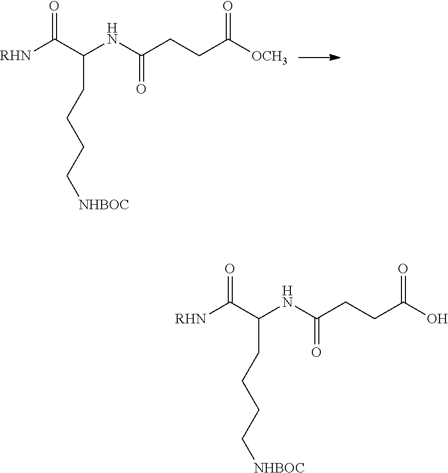

LiOH monohydrate is added to a solution of the above methyl ester in methanol-water (9:1, 50 mL) and the mixture was stirred overnight. Hydrolysis was monitored by TLC indicated by the disappearance of the ester. At the end of reaction, the mixture is concentrated, diluted with water (50 mL), and the aqueous layer is extracted with EtOAc (10 mL) to remove any trace amounts of unhydrolyzed ester. The aqueous layer is cooled and acidified with conc. HCl to acidic pH (˜2). Solid NaCl (20% by vol) is added to the solution and the mixture was extracted twice with methylene chloride (2×50 mL). The mixture is dried (Na₂SO₄), filtered, concentrated and the resulting acid is used without any further purification.

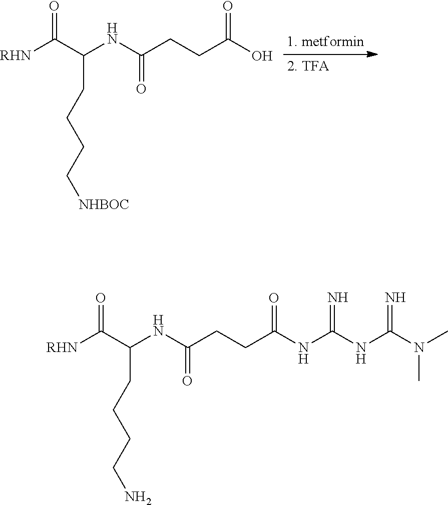

N-Hydroxysuccinimide is added to the above serotonin-lysine carboxylic acid derivative in methylene chloride (15 mL) at 5-0° C. DCC is added in portions over 10 mins period. The resultant suspension is stirred for 16 h and solids are removed under filtration. The filtrate is concentrated to give the active NHS ester, which is used without further purification. The above crude NHS ester is dissolved in THF (10 mL) and lysine-serotoin derivative free base is added followed by the addition of TEA. Addition of few drops of water results in a clear solution which is stirred at 25° C. overnight. The product is purified by column chromatography using methylene chloride-MeOH (0-20% gradient).

### EXAMPLE II

### Objective

The objective of this study was to evaluate Compound (3) (TA1), a novel dual molecular activity treatment for Type 2 diabetes (T2D) in High Fat Diet-Induced Diabetes Mice Model.

### Rationale

Mice were fed whit 60% high-fat diet and then were treated Oraly by Compound (3) (TA1), a novel dual molecular activity, treatment for Type 2 diabetes and to control blood sugar and obesity, with safe and with lower side effects.

### Materials And Methods

### Application

Oral application dosage of Compound (3) (TA1) was used. Compound (3) (TA1) treatments were delivered as a powder ready to use.

### Experimental Model

### Animals

Species/Strain: C57B1/6 mice
Gender/Number/Age: Male, n=42, 3 weeks old
Source: Envigo Ltd., Israel
Body weight: Approximately 10+/−3 gram at study initiation
Acclimation period: NR
Identification: Ear tag and cage card. A cage card contained the study name, animal number and relevant details as to treatment group.

### Animal Management

Housing: Animal handling was performed according to guidelines of the National Institute of Health (NIH) and the Association for Assessment and Accreditation of Laboratory Animal Care (AAALAC). Animals were housed in polyethylene cages measuring 35×30×15 cm, with stainless steel top grill facilitating pelleted food and drinking water in plastic bottle; bedding: steam sterilized clean paddy husk were used and bedding material was changed along with the cage at least twice a week.

Diet: Animals were provided ad libitum a commercial rodent diet, sterilized or 60% HFD. Animals have had free access to acidified autoclaved drinking water obtained from the municipality supply.

Contaminants: The food arrives from the vendor with a certificate of analysis. The water was treated as above.

Environment conditions: Animals were housed in IVC cages in dedicated HVAC (Heat, Ventilation, Air, Conditioning) animal facility at temperature from 22±2° C. and RH (Relative Humidity) of 55±15%. Temperature and humidity were monitored continuously. The facility had no exposure to outside light, and it was maintained on automatic alternating cycles of 12 hours of light and 12 hours of dark.

Randomization: Animals were allocated randomly into the study groups.

Route of administration: IV is the intended route for clinical use, hence the same route was chosen in this study.

### Veterinary Care

Animals were inspected upon arrival; only healthy animals were admitted to the facility and acclimatized for the study. Animals were inspected daily for any signs of morbidity or mortality.

### Justification for Number of Animals

The study included 3 groups of 12 animals in each group. 12 animals in each treatment group were a minimal number of animals that presented significance in the data analysis, enabled to draw conclusions from the experiment.

The same number of animals (n=12) were used in the control groups, to reach the same variability as in the treatment groups.

5 animals served as a normal group, and received normal food and no treatment.

TABLE 1
Group Designation Table

| | | | | Dose starting | |
|---|---|---|---|---|---|
| Group | N | Diet | Treatment | week 7 | Endpoint |
| 1 | 12 | HFD | TA1 - 115 mg/kg | QD for 14 days | 9 weeks |
| 2 | 12 | HFD | TA1 - 346 mg/kg | QD for 14 days | 9 weeks |
| 3 | 12 | HFD | N/A | N/A | 9 weeks |
| 4 | 5 | R Food | N/A | N/A | 9 weeks |

### Experimental Procedures

42 mice, 3 weeks old, arrived at the facility and were divided into 4 groups.
Groups 1-3 received 60% fortified food (HFD)—from study day 1 or the entire duration of the experiment.
Group 4 received a commercial rodent diet.
The animals were monitored visual daily to assess their general condition from day 0 until the end of the experiment. And during all the days of the experiment, no abnormal findings were observed in the animals.
The animals were tested once a week for blood glucose measurement during the experiment.
At 12 weeks of age, the animals began receiving oral treatment twice a day for two weeks.
At the end of the two weeks (14 weeks of age), the animals were fasted overnight (12 hours).
After fasting overnight (12 hours), the animals began an OGTT test (oral glucose tolerance test) after receiving 2 g/kg glucose orally. 30 uL of whole blood will be collected at T0, T30, T60, T120 and 180 min after glucose administration for glucose Measurement and serum production.

### Study Termination

All animals were sacrificed immediately after the last test (180 minutes) by CO₂. There were no signs requiring early termination.

### Results

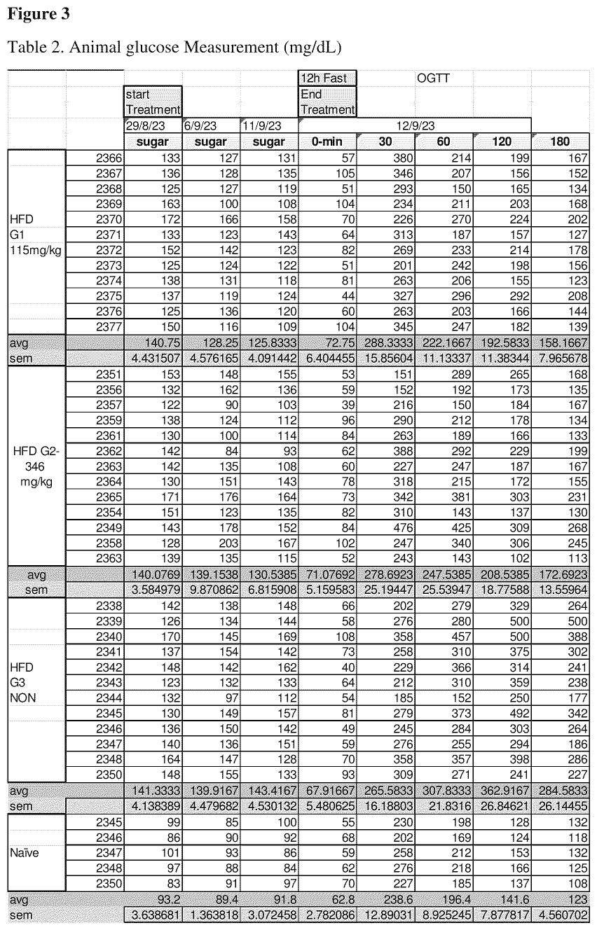

FIG. 3 provides Table 2 showing animal glucose measurement data (mg/dL).

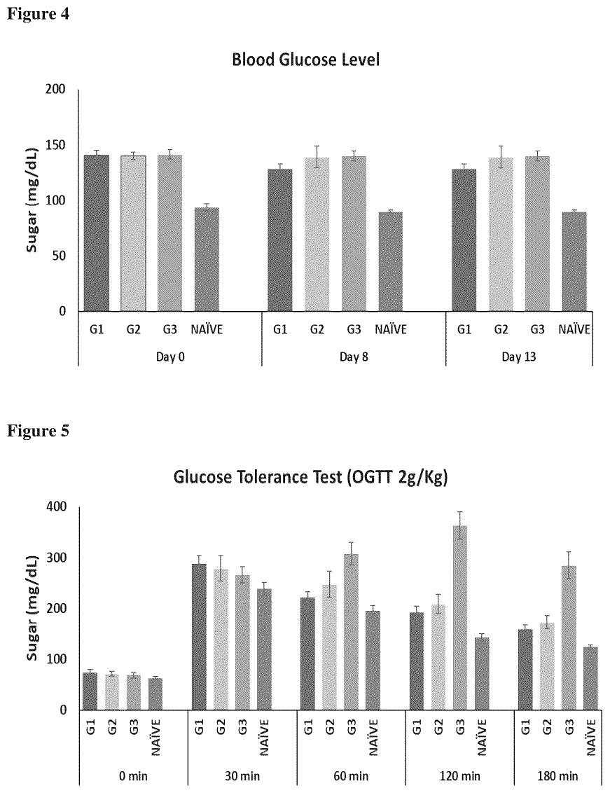

FIG. 4 depicts blood glucose level results.

FIG. 5 depicts glucose tolerance test (OGTT 2 g/kg).

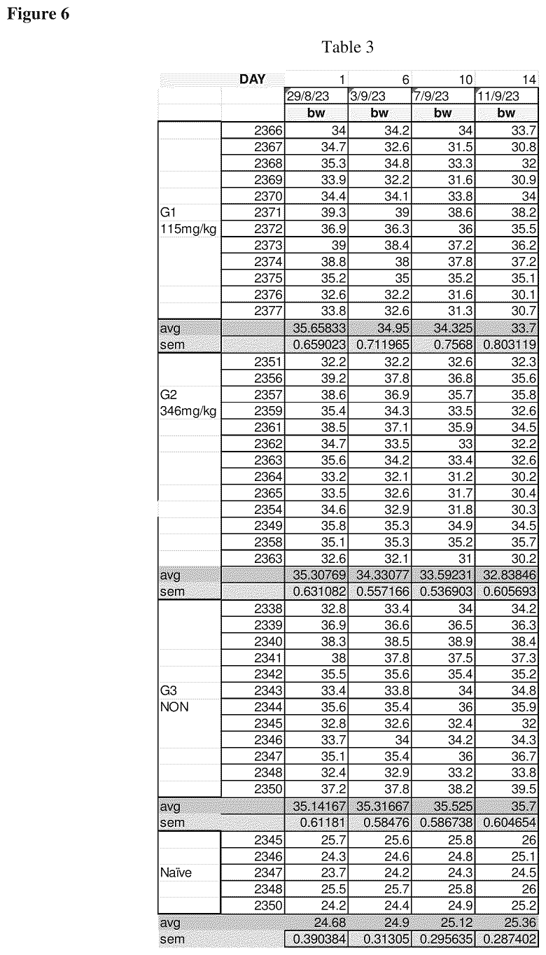

FIG. 6 provides Table 3 showing measurement data.

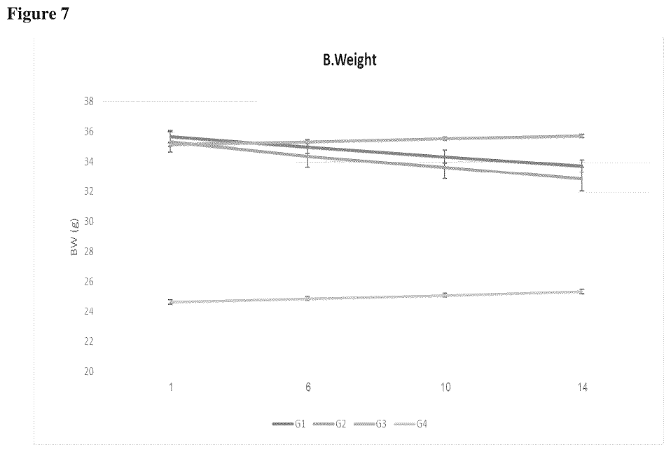

FIG. 7 depicts B. weight results.

The results showed a drop in weight of 5-9% dose-dependent and a drop in sugar level.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A compound represented by formula (I) 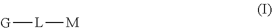
wherein
G is NRₐR_{b}—C(NH)—, a fenfluramine moiety, or a serotonin moiety;
M is —NH—C(NH)—NH—C(NH)—N(CH₃)₂;
L is a linker selected from the group consisting of a bond, —C(O)—(CH₂)ₘ-T-(CH₂)n—C(O)—, -T-C(O)—CH₂—CH₂—C(O)—, —C(O)—CH₂—CH₂—C(O)-T-, —C(O)—CH₂—CH₂—C(O)-T-C(O)—CH₂—CH₂—C(O)—, and a 3-cyclobutene-1,2-dione moiety;
T is a bond, —X₁—CH₂—CH₂—X₂—, an amino acid moiety, a heterocycloalkyl moiety, a hydroxybenzoyl moiety, or an aminophenol moiety;
X₁ and X₂ are each O or NR_{c};
Rₐ, R_{b}, and R_{c} are each independently H, an alkyl group, or an acyl group, and
m and n are each 1, 2, or 3,
or a pharmaceutically acceptable salt thereof.

2. The compound according to claim 1, wherein L is a bond, —C(O)—CH₂—CH₂—C(O)—, a 3-cyclobutene-1,2-dione moiety, or —C(O)—(CH₂)ₘ-T-(CH₂)ₙ—C(O) —.

3. The compound according to claim 2, wherein m is 1 and n is 1.

4. The compound according to claim 1, wherein L is 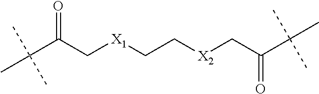
wherein X; and X₂ are O or NH.

5. The compound according to claim 1, wherein L is —C(O)—CH₂—OCH₂CH₂O—CH₂—C(O)—, —C(O)—CH₂—NHCH₂CH₂NH—CH₂—C(O)—, —C(O)—CH₂-NHCH₂CH₂O—CH₂—C(O)—, or —C(O)—CH₂—OCH₂CH₂NH—CH₂—C(O)—, and wherein T is a piperazine moiety or a piperidine moiety.

6. The compound according to claim 1, wherein L is 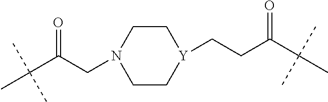
wherein Y is N or CH.

7. The compound according to claim 1, wherein L is -T- C(O)—(CH₂)ₘ—(CH₂)ₙ—C(O)—, and wherein T is an amino acid moiety.

8. The compound according to claim 7, wherein said amino acid moiety is a lysine moiety or an arginine moiety.

9. The compound according to claim 1, wherein L is

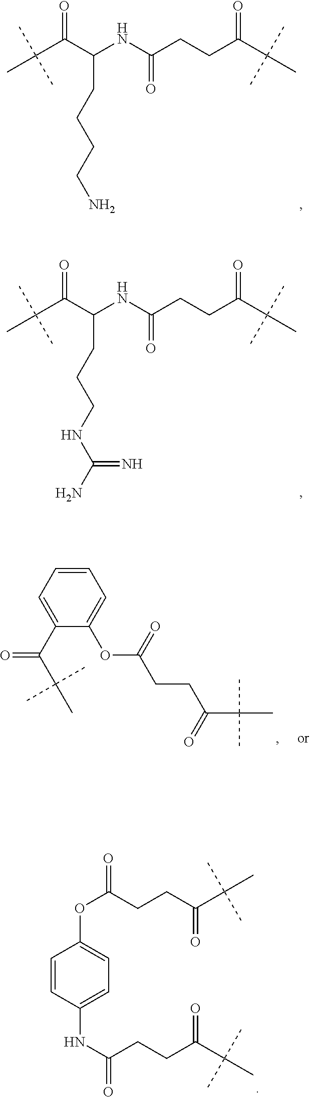

10. The compound according to claim 1, wherein L is -T-C(O)—CH₂—CH₂—C(O)—, and wherein T is a hydroxybenzoyl moiety.

11. The compound according to claim 1, wherein L is —C(O)—CH₂—CH₂—C(O)-T-C(O)—CH₂—CH₂—C(O)—, and wherein T is an aminophenol moiety.

12. The compound according to claim 1, wherein G is NRₐR_{b}—C(NH)—, a fenfluramine moiety, or a serotonin moiety, and wherein Rₐ is H and R_{b} is H or acetyl.

13. The compound according to claim 1, wherein the compound of formula (I) is represented by a compound of formula (II) 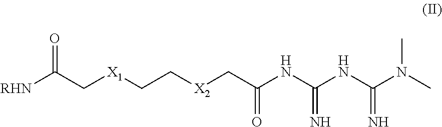
wherein RNH is a fenfluramine moiety or a serotonin moiety, and wherein X₁ and X₂ are O or NH.

14. The compound according to claim 1, wherein the compound of formula (I) is represented by a compound of formula (III) 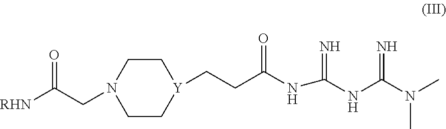
wherein RNH is a fenfluramine moiety or a serotonin moiety, and wherein Y is N or CH.

15. The compound according to claim 1, wherein the compound of formula (I) is represented by a compound of formula (IV) 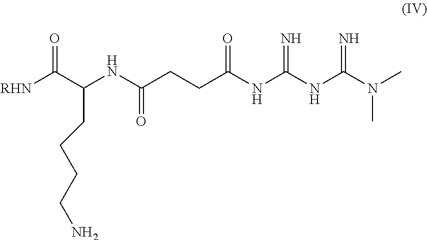
wherein RNH is a fenfluramine moiety or a serotonin moiety.

16. The compound according to claim 1, wherein the compound of formula (I) is represented by a compound of formula (V) 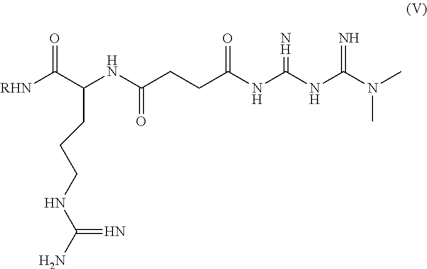
wherein RNH is a fenfluramine moiety or a serotonin moiety.

17. The compound according to claim 1, wherein said compound is

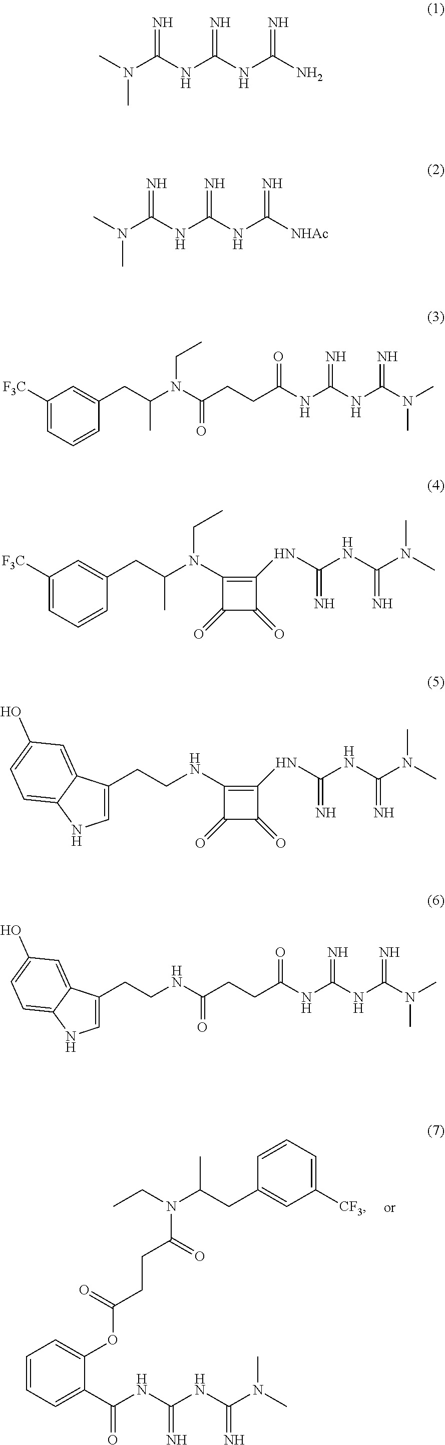

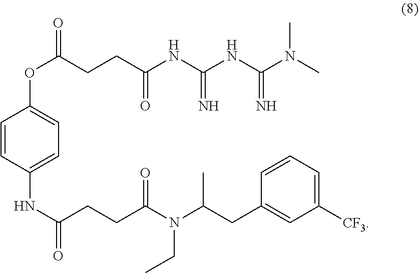 [SMILES: CCN(C(=O)CCC(=O)Nc1ccc(OC(=O)CCC(=O)NC(=N)NC(=N)N(C)C)cc1)C(C)Cc1cccc(C(F)(F)F)c1]

18. A pharmaceutical composition comprising a compound according to claim 1 and a pharmaceutically acceptable carrier.

19. A method of treating Type 2 diabetes in a subject in need thereof, comprising administering to the subject a compound according to claim 1.

20. A method of treating obesity or inducing weight reduction in a subject in need thereof, comprising administering to the subject a compound according to claim 1.
